(19) United States
(12) Patent Application Publication
(10) Pub. No.: US 2026/0207212 A1
(43) Pub. Date: Jul. 23, 2026

(54) INTRAVASCULAR LITHOTRIPSY DEVICES, SYSTEMS, AND METHODS

(71) Applicant: Boston Scientific Scimed, Inc., Maple Grove, MN (US)

(72) Inventors: Javier Palomar-Moreno, Galway (IE); Olena Pernatiy, Galway (IE); Anthony Malone, Galway (IE)

(73) Assignee: Boston Scientific Scimed, Inc., Maple Grove, MN (US)

(21) Appl. No.: 19/382,866

(22) Filed: Nov. 7, 2025

Related U.S. Application Data
(60) Provisional application No. 63/718,243, filed on Nov. 8, 2024.

(51) Int. Cl. A61B 17/22; A61B 17/00; A61M 25/10

(52) U.S. Cl. CPC A61B 17/22022; A61M 25/1002; A61B 2017/00141; A61B 2017/00557; A61B 2017/00778; A61B 2017/00893; A61B 2017/22025; A61B 2017/22065; A61M 2025/105; A61M 2205/0238; A61M 2210/12

(57) ABSTRACT

An intravascular lithotripsy device may include an elongate shaft, at least one wave generator, and at least one expandable member extending around the at least on wave generator. The at least one expandable member may be inflatable with a fluid. The at least one expandable member may have a plurality of lobes, including a first lobe and a second lobe longitudinally spaced from the first lobe. The at least one expandable member may have a sinusoidal shape. A power generator may be electrically coupled with the at least one wave generator and configured to actuate the at least one wave generator to initiate a wave within the at least one expandable member.

### CROSS-REFERENCE TO RELATED APPLICATIONS

This application claims the benefit of priority under 35 U.S.C. § 119 to U.S. Provisional Application No. 63/718,243, filed Nov. 8, 2024, the entire disclosure of which is hereby incorporated by reference herein for all purposes.

### TECHNICAL FIELD

The present disclosure pertains to lithotripsy devices, systems, and methods. More specifically, the present disclosure relates to intravascular lithotripsy devices, systems, and methods configured to treat plaque and/or other lesions in vasculature of a subject.

### BACKGROUND

A wide variety of intracorporeal and extracorporeal medical devices and systems have been developed for medical use, for example, in vasculature procedures and/or for vasculature treatments. Some of these devices and systems include guidewires, catheters, catheter systems, pump devices, lithotripsy devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including intravascular lithotripsy devices.

In a first example, a medical device may include an elongate shaft, at least one wave generator, and at least one expandable member extending around the at least one wave generator, and wherein the at least one expandable member has a plurality of lobes including a first lobe and a second lobe longitudinally spaced from the first lobe.

Alternatively or additionally to any of the examples above, the at least one expandable member may have a sinusoidal shape when inflated.

Alternatively or additionally to any of the examples above, a wave generator of the at least one wave generator may be longitudinally aligned with an apex of one of the first lobe and the second lobe.

Alternatively or additionally to any of the examples above, a first wave generator of the at least one wave generator may be longitudinally aligned with a first apex of the first lobe and a second wave generator of the at least one wave generator may be longitudinally aligned with a second apex of the second lobe.

Alternatively or additionally to any of the examples above, the medical device may further include a ring extending circumferentially around the at least one expandable member between the first lobe and the second lobe, the ring having an outer diameter that is less than an outer diameter of the first lobe, an outer diameter of the second lobe, or the outer diameter of the first lobe and the outer diameter of the second lobe.

Alternatively or additionally to any of the examples above, the medical device may further include a first ring extending circumferentially around the at least one expandable member, a second ring extending circumferentially around the at least one expandable member, a third lobe of the plurality of lobes, the third lobe being longitudinally spaced in a distal direction from the first lobe and the second lobe, and wherein the first ring extends circumferentially around the at least one expandable member between the first lobe and the second lobe and the second ring extends circumferentially around the at least one expandable member between the second lobe and the third lobe.

Alternatively or additionally to any of the examples above, the first lobe may be in fluid communication with the second lobe.

Alternatively or additionally to any of the examples above, the medical device may further include a fluid lumen extending along the elongate shaft, the fluid lumen being in fluid communication with the expandable member.

Alternatively or additionally to any of the examples above, the fluid lumen may be in fluid communication with each of the plurality of lobes.

Alternatively or additionally to any of the examples above, a wave generator of the at least one wave generator may include a piezoelectric transducer.

Alternatively or additionally to any of the examples above, the medical device may further include an antiproliferative drug coupled with an exterior surface of one or more of the plurality of lobes.

Alternatively or additionally to any of the examples above, the antiproliferative drug may be coupled with the exterior surface of the at least one expandable member using a biodegradable polymer.

In a further example, a system may include a catheter comprising an elongate shaft, at least one wave generator, and at least one expandable member extending around the at least one wave generator, wherein the at least one expandable member comprises a sinusoidal shape when inflated, a power generator configured to actuate the at least one wave generator to initiate a wave within the at least one expandable member.

Alternatively or additionally to any of the examples above, the power generator may be configured to apply an alternating current to the at least one wave generator.

Alternatively or additionally to any of the examples above, the at least one expandable member may be coated with an antiproliferative drug bound to an outer surface of the at least one expandable member with an excipient.

Alternatively or additionally to any of the examples above, the power generator may be configured to apply an alternating current to the at least one wave generator at a frequency that causes the wave generated by the at least one wave generator to detach the antiproliferative drug from the outer surface of the at least one expandable member.

Alternatively or additionally to any of the examples above, the catheter comprises a fluid lumen configured to provide fluid to the at least one expandable member.

In a further example, a method for treating a stenosis in a body lumen may include advancing a catheter within the body lumen to a position proximate to the stenosis, the catheter comprising an elongate shaft, at least one wave generator, and at least one expandable member extending around the at least one wave generator and having a plurality of lobes, inflating the at least one expandable member with a fluid so that an outer surface of an apex of at least one of the plurality of lobes contacts the stenosis, and actuating the at least one wave generator to generate at least one wave through the fluid and the apex of the at least one of the plurality of lobes.

Alternatively or additionally to any of the examples above, the at least one expandable member may be coated with an antiproliferative drug, and wherein the at least one wave generated by the at least one wave generator may be configured to detach the antiproliferative drug from the at least one expandable member.

Alternatively or additionally to any of the examples above, the method may further include longitudinally adjusting the catheter to treat different portions of the stenosis sequentially.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative configuration of an intravascular lithotripsy system;

FIG. 2 is a schematic diagram of an illustrative configuration of a controller/power generator and a user interface;

FIG. 3 is a schematic perspective view of a portion of an illustrative configuration of an intravascular lithotripsy device;

FIG. 4 is a schematic side view of a portion of an illustrative configuration of an intravascular lithotripsy device;

FIG. 5 is a schematic cross-section view of the intravascular lithotripsy device depicted in FIG. 4, taken along line 5-5;

FIG. 6 is a schematic diagram of an illustrative method of using an intravascular lithotripsy device; and

FIGS. 7A-7D depict schematic diagrams of an illustrative method of using an intravascular lithotripsy device, with a vessel shown in cross-section and a portion of the intravascular lithotripsy device shown from a side view.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Intravascular lithotripsy (IVL) is a technique used for treating calcified lesions in blood vessels. IVL may be a minimally invasive procedure that employs catheters with an integrated balloon containing one or more emitters or wave generators to generate sonic pressure waves that selectively impact hardened calcium deposits within vessel walls. Previous approaches for treating calcified lesions include angioplasty, cutting balloons, and rotational atherectomy, which have limitations in effectively modifying heavily calcified plaques and/or risk vessel damage.

The present disclosure provides medical devices and systems (e.g., an IVL device and system) that enhance treatment of calcified lesions (e.g., plaque) or a stenosis in vessels over existing treatments. The medical devices may include an elongate shaft, at least one wave generator, and at least one expandable member extending around the at least one wave generator. The expandable member may have multiple lobes longitudinally spaced from one another. In some examples, an outer contour of the expandable member may have or define a longitudinal sinusoidal shape when expanded and/or otherwise (e.g., when not expanded). This sinusoidal design of the expandable member may be configured to transfer forces generated by the at least one wave generator to a target area or location to modify calcified lesions or a stenosis at the target area. The expandable member may include or may be covered with an antiproliferative drug coupled with a surface of the expandable member via an excipient.

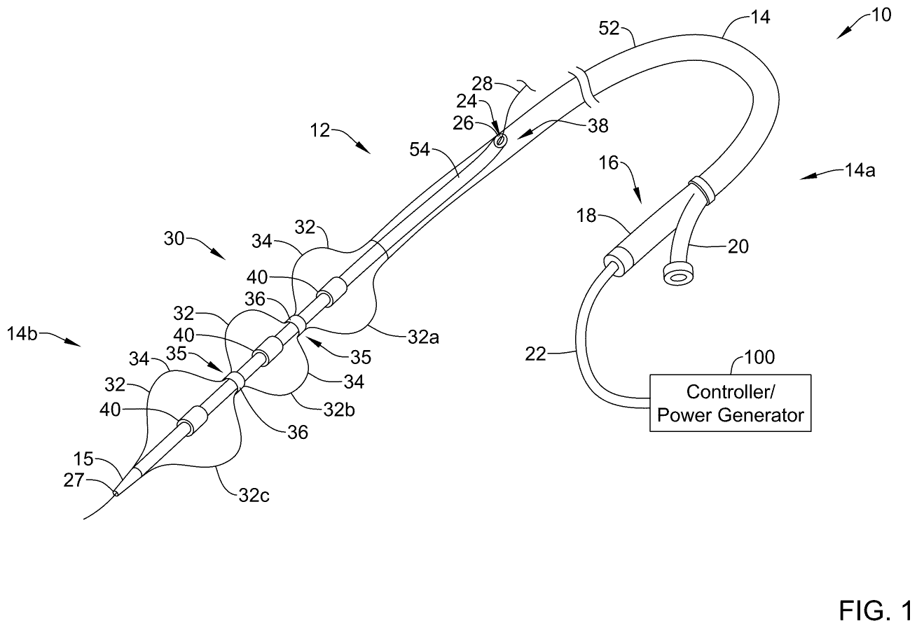

FIG. 1 is a schematic perspective view of an illustrative configuration of an intravascular lithotripsy (IVL) system 10. The IVL system 10 may include any suitable components for performing IVL treatments on a vessel of a subject (e.g., a patient). For example, the IVL system 10 may include, among other suitable components, a medical device 12 (e.g., a catheter, etc.) and a controller/power generator 100. The medical device 12 may include, among other suitable components, an elongate shaft 14, one or more emitters or wave generators 40, and one or more expandable members 30 (e.g., a balloon, etc.) As depicted in FIG. 1, the medical device 12 may be coupled with the controller/power generator 100 using a power connector 22 (e.g., a wired or wireless electrical connector).

As discussed, the medical device 12 may include the elongate shaft 14, the one or more wave generators 40, and the one or more expandable members 30. In some examples, the one or more expandable members 30 may be part of and/or located at a distal portion of the elongate shaft 14 and a hub 16 may be part of and/or located at a proximal portion of the elongate shaft 14. The medical device 12 may terminate at a distal tip 15 positioned distal of and/or at a distal end of the one or more expandable members 30. The distal tip 15 may be part of or a component separate from the elongate shaft 14. Although other suitable configurations are contemplated, the distal tip 15 may have an outer surface that is tapered in a distal direction.

The hub 16 may be part of the elongate shaft 14 or coupled with and a component separate from the elongate shaft 14. In some examples, the hub 16 may have one or more ports. For example, the hub 16 may have an electrical port 18, a fluid port 20, a guidewire port, and/or one or more other suitable ports. The ports of the hub 16 may be in communication with one or more lumens of the elongate shaft 14. In some examples, the electrical port 18 may be configured to be permanently coupled to the power connector 22 or may be configured to releasably receive the power connector 22 coupled with or couplable with the controller/power generator 100.

The elongate shaft 14 may have any suitable configuration. In some examples, the elongate shaft 14 may have a proximal end portion 14a and a distal end portion 14b and may include and/or entirely or at least partially define one or more lumens. For example, the elongate shaft 14 may include a guidewire lumen 24, a fluid lumen 38, a wire lumen, and/or other suitable lumens, which may each extend through the proximal end portion 14a and/or the distal end portion 14b of the elongate shaft 14.

The guidewire lumen 24 may have any suitable configuration. For example, the guidewire lumen 24 may extend from a guidewire port 26 at a proximal end to a distal opening 27 at a distal end of the guidewire lumen 24. The guidewire port 26 may be a side opening in the elongate shaft 14 between the hub 16 and the expandable member 30 (e.g., the guidewire port 26 may be configured as a rapid exchange guidewire port), as depicted for example in FIG. 1. Alternatively or additionally, the guidewire port 26 may be located in the hub 16 (e.g., the guidewire port 26 may be configured as an over-the-wire guidewire port). The guidewire port 26, guidewire lumen 24, and the distal opening 27 may be configured to receive a guidewire 28 and facilitate advancing the medical device 12 in distal and proximal directions over the guidewire 28. Although the medical device 12 is discussed herein as including the guidewire lumen 24, the guidewire lumen 24 may be omitted.

The fluid lumen 38 may have any suitable configuration. For example, the fluid lumen 38 may be configured to extend from a fluid port 20 to the expandable member 30, such that fluid may be received at the fluid port 20, pass through the fluid lumen 38, and be delivered to the expandable member 30 to expand or inflate the expandable member 30. In some examples, the fluid port 20 may be located at the hub 16 and configured to couple with a fluid source configured to provide fluid to and/or remove fluid from the fluid lumen 38 and/or the expandable member 30.

The lumens of the elongate shaft 14 may be formed in any suitable manner. In some examples, the lumens of the elongate shaft 14 may be formed from one or more tubes, an extrusion defining the lumens, and/or formed in one or more other suitable manners. In one example, the elongate shaft 14 may include a first tube 52 (e.g., an outer tube) defining the fluid lumen 38 and a second tube 54 (e.g., an inner tube, located at least partially within the first tube 52) entirely or at least partially defining the guidewire lumen 24 and extending between the guidewire port 26 and the distal opening 27. The elongate shaft 14 may include additional and/or alternative configurations of tubes and/or lumens.

The one or more expandable members 30 may have any suitable configuration. In some examples, the one or more expandable members 30 may include one or more balloons configured to expand in response to fluid being delivered to the balloons. The one or more expandable members 30 may be part of or coupled with the elongate shaft 14. In one example, a proximal end of the expandable member 30 may be coupled with a distal end or distal portion of the elongate shaft 14 such that the fluid lumen 38 is in fluid communication with an interior of the one or more expandable members 30. In the example, a distal end may be coupled with the distal tip 15 and/or a tube defining the guidewire lumen 24 to close the one or more expandable members 30 at the distal end thereof to maintain fluid within the one or more expandable members 30.

The one or more expandable members 30 may have any suitable shape. For example, the shape of the one or more expandable members 30 may be or may include a cylindrical shape, a bulbous shape, a sinusoidal shape, and/or any other suitable shape. In one example configuration of an expandable member 30, the expandable member 30 may be shaped so as to extend circumferentially around the guidewire lumen 24 and define one or more lobes 32 fluidly coupled with the fluid lumen 38. In some examples, the guidewire lumen 24 may be omitted and the one or more lobes may extend circumferentially around a central longitudinal axis of the elongate shaft 14 or the medical device 12. Each of the one or more lobes 32 may define a peak or an apex 34 and taper toward the central longitudinal axis of the elongate shaft 14 or the medical device 12 to either side of the peak or apex 34.

The expandable members 30 and/or features of the expandable members 30 may have any suitable length. For example, the peak or apex 34 of the expandable members 30 may have any suitable longitudinal length. In one example, the peak or apex 34 of the expandable members 30 may have a length that extends an entire length of one of the expandable members 30. In one example, the peak or apex 34 of the expandable members 30 may have a length that is configured to extend at least a length of a lesion or target location or area. When the expandable members 30 defines two or more lobes 32, the expandable members 30 may have any suitable length between peaks or apices 34 and/or between valleys 35.

When the expandable member 30 defines two or more lobes 32, the two or more lobes 32 may be fluidly coupled and/or longitudinally spaced from one another. The two or more lobes 32 of the expandable member 30 may also define the peak or apex 34 at each lobe 32 and a valley 35 between each lobe 32. In one example, the expandable member 30 may include a first lobe 32a, a second lobe 32b longitudinally spaced in a distal direction from the first lobe 32a, and a third lobe 32c longitudinally spaced in a distal direction from the second lobe 32b, but other suitable configurations are contemplated. When the expandable member 30 defines two or more lobes 32, the expandable member 30 may have an exterior surface with a sinusoidal shape, when expanded or inflated as depicted for example in FIG. 1 and/or when not expanded or inflated.

The shape of the lobe 32 with the apex or peak 34 and the tapering ends or the valleys 35 may direct acoustic pressure waves generated by the wave generator 40 associated with the lobe 32 through the expandable member 30 at or around the apex or peak 34 of the lobe 32. For example, although the acoustic pressure waves may be able to move through the material of the expandable member 30, the material and shape of the lobe 32 may urge the acoustic pressure waves to leave the expandable member 30 at or proximate the apex or leak 34 of the lobe 32. Directing the acoustic pressure waves through the expandable member 30 at or around the apex or peak 34 of the lobe 32 may amplify forces of the generated acoustic pressure waves to more efficiently modify calcium or plaque build-up at or proximate the target location (e.g., a location at which the calcified lesion or stenosis is located and the apex or peak 34 is positioned in a vessel) than when waves from the wave generators propagate radially outward from wave generators 40 freely (e.g., when one or more wave generators are positioned within an elongate cylindrical balloon).

The one or more expandable members 30 may be formed from any suitable material. For example, the one or more expandable member 30 may be formed from materials including, but not limited to, non-compliant materials, semi-compliant materials, polymers, metals, alloys, polyamides, polyesters, polyethers, polyacrylates, nylons, polyethylene terephthalate (PET), polyurethanes, polyolefins (e.g., HDPE, etc.), and/or other suitable materials. Although the material of the one or more expandable members 30 is depicted as being optically transparent in FIG. 1 to show components of the medical device 12 within the one or more expandable members 30, other suitable configurations are contemplated in which the material of the one or more expandable members 30 is optically semi-transparent and/or opaque.

The medical device 12 may include one or more annular members 36 extending around (e.g., circumferentially around) the one or more expandable members 30. The one or more annular members 36 may be configured to facilitate maintaining a desired configuration (e.g., a desired geometry, such as shape and size) of the one or more expandable members 30. In some examples, when the expandable member 30 defines two or more lobes 32, an annular member 36 may be positioned around the expandable member 30 at regular intervals (e.g., at the valley 35 between two lobes 32) to facilitate maintaining a shape of the expandable member 30 that defines the two or more lobes 32. In some examples, the annular member 36 may have an inner diameter sufficient to allow each lobe 32 to be in fluid communication with one another and/or the fluid lumen 38. In some examples, the annular member(s) 36 may have an outer diameter that is less than an outer diameter of all of or at least one of the lobes 32 of the expandable member 30. In some examples, the annular member(s) 36 may be omitted and the expandable member 30 may be configured to maintain a desired shape in one or more other suitable manners including, but not limited to, from the configuration of the material used to form the expandable member 30.

The annular member 36 may have any suitable configuration and may be formed from any suitable material. In some examples, the annular member 36 may be a solid ring, a flexible ring, a strap, a belt, and/or other suitable annular configuration. In some examples, the annular member 36 may be formed from a polymer material, a metal material, a fabric material, a natural fiber material, a radiopaque material, and/or other suitable materials.

The medical device 12 may include one or more wave generators 40 configured to create an acoustic pressure wave in the fluid within the expandable member 30. In some examples, the fluid within the expandable member 30 may be used to expand or inflate the expandable member 30. In some examples, the fluid (e.g., contrast media, saline, etc.) used to inflate the expandable member 30 may be a conductive fluid that conducts the wave generated or initiated by the wave generator 40 to and through the expandable member 30 to a target area or location at a wall of a vessel of a subject.

The wave generators 40 may have any suitable configuration for generating an acoustic pressure wave in and/or through the fluid within the expandable member 30 including, but not limited to, a piezoelectric transducer that creates or initiates a pressure wave in response to being actuated with electrical signals, an optical transducer that creates or initiates a pressure wave in response to being actuated with an optical signal (e.g., a laser and/or other suitable optical signal), a spark generator that creates or initiates a pressure wave in response to electrical signals, an acoustic field application that produces cavitation (formation, growth, and implosive collapse of bubbles with high-frequency ultrasonic waves), localized heating applied with microheaters or focused microwave energy that vaporizes microbubbles within the fluid and/or other suitable high frequency wave generators 40. In one example configuration of the wave generators 40, the wave generators 40 may include one or more piezoelectric transducers electrically coupled with the controller/power generator 100 via one or more conductors or electrodes (not shown in FIG. 1) to receive electrical signals and translate the electrical signals into mechanical movements of the piezoelectric transducer that generates acoustic pressure waves in the fluid expanding the expandable member 30. In one example configuration of the wave generators 40, the wave generators 40 may be configured to facilitate focusing an optical signal, such as a laser beam, to heat a small region within the fluid, causing rapid vaporization of microbubbles. The sudden expansion and collapse of these bubbles may generate acoustic pressure waves. Other suitable configurations of the wave generators 40 are contemplated.

The expandable member 30 may be configured to extend around the one or more wave generators 40. In one example, the one or more wave generators 40 may be located at an outer surface of the guidewire lumen 34 between the guidewire lumen 34 and the expandable member 30, but other suitable positions of the wave generators 40 relative to the expandable member 30 are contemplated. When the expandable member 30 defines one or more lobes 32, the peak or apex 34 of each of the lobes 32 may be axially or longitudinally aligned with a wave generator 40 of the one or more wave generators 40 (e.g., the peak or apex 34 and an associated wave generator 40 may share a plane perpendicular to a longitudinal axis of the medical device 12). The positioning of the wave generator 40 at a location aligned with a peak or apex 34 of the lobe 32 may facilitate concentrating and/or directing waves generated by the wave generator 40 to or at a target area or location along a vessel wall of a subject. In one example configuration of the medical device 12, a first wave generator 40 may be aligned with the peak or apex 34 of the first lobe 32a extending circumferentially around the first wave generator 40, a second wave generator 40 may be aligned with the peak or apex 34 of the second lobe 32b extending circumferentially around the second wave generator 40, and a third wave generator 40 may be aligned with the peak or apex 34 of the third lobe 32c extending circumferentially around the third wave generator 40, as depicted for example in FIG. 1. The alignment of the wave generator 40 with the apex or peak 34 of the lobe 32 may facilitate providing the acoustic pressure waves through the expandable member 30 at or around the apex or peak 34 to more efficiently modify calcium or plaque build-up at or proximate the target location than when the wave generators may not be aligned with the apex or peak 34.

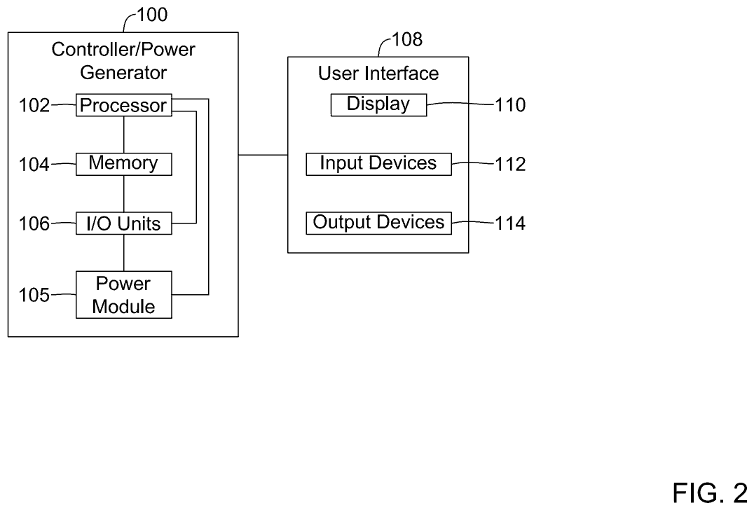

FIG. 2 depicts a schematic diagram of an illustrative configuration of the controller/power generator 100 (e.g., computing device) and a user interface 108 of the IVL system 10. The controller/power generator 100 may be and/or may include any suitable computing device configured to process data of or for the IVL system 10 and provide power to the one or more wave generators 40 via, in some examples, the power connector 22 and electrical conductors extending between the power connector 22 and the one or more wave generators 40. In some cases, one or more components of the IVL system 10 may be incorporated into the controller/power generator 100 and/or the user interface 108. Further, one or more components of the IVL system 10 may incorporate one or more computing devices similar to or having components similar to the controller/power generator 100 and/or the user interface 108.

The controller/power generator 100 may be configured to facilitate operation of the IVL system 10. The controller/power generator 100, in some cases, may be configured to monitor and/or control an amount of power and/or a frequency of power applied to the one or more wave generators 40 according to a control program (e.g., a preconfigured control program, a control program configured in real time, etc.) The control program may be based on past or present user interactions with the user interface 108, and/or in response to values from one or more sensors and/or values of one or more monitored metrics or parameters reaching or going beyond a threshold value. In some examples, the controller/power generator 100 may be configured to provide electrical current pulses (e.g., alternating current (AC) or direct current (DC)), high voltage pulses, optical pulses, and/or other suitable pulses of power to the wave generators 40, but other suitable configurations are contemplated. The controller/power generator 100 may be in communication with a wall power source, a battery power source, a renewable energy power source, and/or other suitable source of power.

The controller/power generator 100 may communicate with a remote server or other suitable computing device. When the controller/power generator 100, or at least a part of the controller/power generator 100, is a component separate from a structure of the medical device 12, the controller/power generator 100 may communicate with electronic components of the IVL system 10 over one or more wired or wireless connections or networks (e.g., LANs and/or WANs) (e.g., via the power connector 22 and/or other suitable input/output components).

The controller/power generator 100 may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs) or System On Chips (SOCs), software, hardware, firmware, or any combination of these and/or other components. Although the controller/power generator 100 may be referred to herein in the singular, the controller/power generator 100 may be implemented in multiple instances, distributed across multiple computing devices, instantiated within multiple virtual machines, and/or the like.

The illustrative controller/power generator 100 may include, among other suitable components, one or more processors 102, memory 104, one or more power modules 105, and/or one or more input/output (I/O) units 106. Example other suitable components of the controller/power generator 100 that are not specifically depicted in FIG. 2 may include, but are not limited to, communication components, a touch screen, selectable buttons, a housing, and/or other suitable components of a controller. As discussed above, one or more components of the controller/power generator 100 may be separate from the components of the IVL system 10 and/or incorporated into the components of the IVL system 10.

The processor 102 of the controller/power generator 100 may include a single processor or more than one processor working individually or with one another. The processor 102 may be configured to receive and execute instructions, including instructions that may be loaded into the memory 104 and/or other suitable memory. Example components of the processor 102 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 104 of the controller/power generator 100 may include a single memory component or more than one memory component each working individually or with one another. Example types of memory 104 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 104 may be or may include a non-transitory computer readable medium. The memory 104 may include instructions stored in transitory and/or non-transitory state on a computer readable medium that may be executable by the processor 102 to cause the processor 102 to perform one or more of the methods and/or techniques described herein.

The power module 105 may include any suitable component configured to facilitate providing power to the one or more wave generators 40. Example suitable components of the power module 105 may include, but are not limited to, a DC-DC converter, a DC-AC converter, an optical converter (e.g., a component configured to convert electrical power to into optical power (e.g., a light or laser beam)), a high voltage capacitor, a transistor switch, a power measurement unit (e.g., voltage measurement unit, current measurement unit, optical power measurement unit, and/o rother suitable measurement unit), a device identification unit (e.g., a unit that identifies whether the medical device is compatible with the controller/power generator 100), and/or other suitable components.

The I/O units 106 of the controller/power generator 100 may include a single I/O component or more than one I/O component each working individually or with one another. Example I/O units 106 may be or may include any suitable types of mechanical communication hardware, electrical communication hardware, optical communication hardware, and/or software including, but not limited to, power input ports to receive power from a power source, power output ports to provide power to the medical device 12, device ports for coupling with the medical device 12, communication ports configured to communicate with electronic components of the IVL system 10 and/or with other suitable computing devices or systems. Example types of I/O units 106 may include, but are not limited to, wired power components, wired optical components, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless power components, wireless optical components, wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 106.

The user interface 108 may be configured to communicate with the controller/power generator 100 via one or more wired or wireless connections. The user interface 108 may include one or more display devices 110, one or more input devices 112, one or more output devices 114, and/or one or more other suitable features.

The display device 110 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 112 may be and/or may include any suitable components and/or features for receiving user input via the user interface. Example input device(s) 160 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) 114 may be and/or may include any suitable components and/or features for providing information and/or data to users and/or other computing components. Example output device(s) 162 include, but are not limited to, displays, speakers, vibration systems, tactile feedback systems, optical outputs, cables, lights, and/or other suitable output devices.

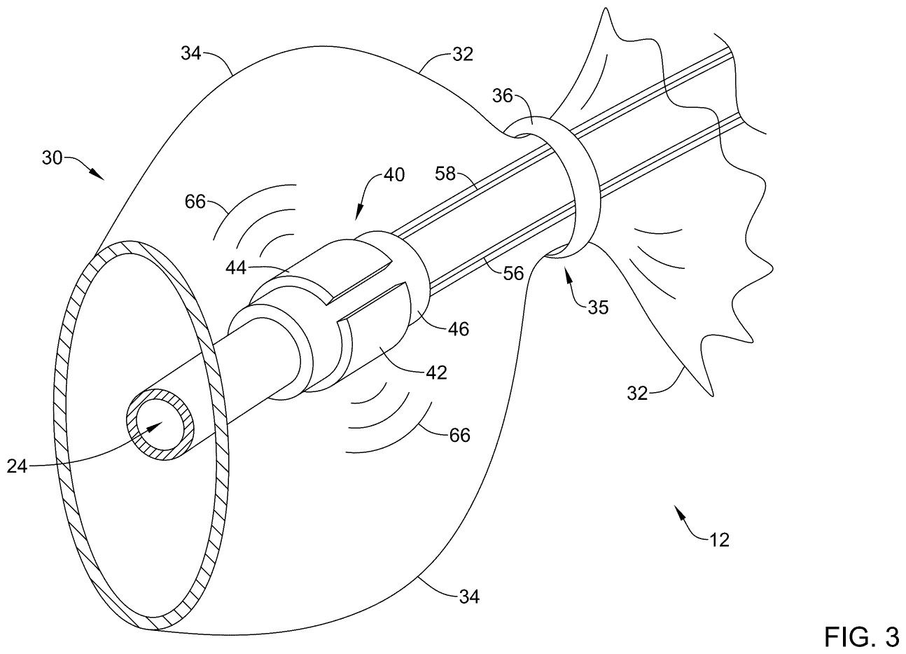

FIG. 3 depicts a schematic perspective view of a portion of an illustrative configuration of the medical device 12 depicting a lobe 32 of the one or more lobes 32 of the one or more expandable members 30 and an associated wave generator 40. As discussed, the associated wave generator 40 may be longitudinally aligned with the apex or peak 34 extending circumferentially around the lobe 32.

As discussed, the wave generator 40 may take on any suitable configuration. For example, the wave generator 40 may be configured to create an acoustic wave in the fluid inflating or expanding the expandable member 30 in response to receiving electrical power from the controller/power generator 100, vibrations (e.g., high frequency vibrations) in response to receiving electrical power from the controller/power generator 100, heat in response to receiving optical power from the controller/power generator 100, and/or via one or more other suitable pressure wave generation mechanisms. In one example, the wave generator 40 may be configured to create an acoustic pressure wave in the fluid inflating or expanding the expandable member 30 via vibrations of a piezoelectric transducer 46 in response to receiving electrical power from the controller/power generator 100 via one or more electrically conductive wires (e.g., insulated wires and/or other suitable wires), as depicted for example in FIG. 3.

When the wave generator 40 is configured to create acoustic pressure waves, the wave generator may include the piezoelectric transducer 46, a first electrode 42 (e.g., a cathode or a source electrode), and a second electrode 44 (e.g., an anode or a return electrode). In some examples, the first electrode 42 may be coupled with a first electrically conductive wire 56 configured to provide current (e.g., electrically charged carrier movement) to the wave generator 40 from the controller/power generator 100 via the power connector 22 and the second electrode 44 may be coupled with a second electrically conductive wire 58 configured to return current to the controller/power generator 100 or other component of the IVL system 10 via the power connector 22. In some examples, the first wire 56 may be part of the first electrode 42 and/or the second wire 58 may be part of the second electrode 44.

Although the medical device 12 depicted in FIG. 3 includes only the first wire 56 and the second wire 58 that terminate at the depicted wave generator 40, other suitable wires, wire configurations, and/or power communication components are contemplated. In some examples, when there are multiple wave generators 40, the first wire 56 and the second wire 58 may extend to each of the wave generators 40 such that the multiple wave generators 40 in communication with the first wire 56 and the second wire 58 (e.g., initiate acoustic pressure waves at the same or nearly the same time. In some examples, when there are multiple wave generators 40, the medical device 12 may include wires (e.g., wire pairs or individual wires) in communication with the controller/power generator 100 that are in addition to the first wire 56 and the second wire 58 such that the wave generators 40 coupled with the additional wires may operate independent of at least one or more other wave generators 40.

The piezoelectric transducer 46 may be formed from any suitable material. Example suitable materials for the piezoelectric transducer 46 include, but are not limited to, crystalline materials, ceramic materials, polymeric materials, lead zirconate titanate (PZT), barium titanate, lead titanate, gallium nitride, zinc oxide, quartz, natural crystals, quartz, Rochelle Salt, Barium Titanate (BaTiO3), Polyvinylidene Fluoride (PVDF), Lithium Niobate (LiNbO3), Potassium Niobate (KNbO3), and/or other suitable piezoelectric materials. In one example, the piezoelectric transducer 46 may be formed from a crystalline material, but other suitable configurations are contemplated.

In operation, a current (e.g., an alternating current or other suitable current) may be provided to the piezoelectric transducer 46 of the wave generator 40 via the first wire 56 and the first electrode 42, the current may pass through the piezoelectric transducer 46 to the second electrode 44 and the second wire 58. In response to the current passing through the piezoelectric transducer 46, the piezoelectric transducer 46 may oscillate and change shape and/or size. When pulses of current are sent through the piezoelectric transducer 46, the piezoelectric transducer 46 may generate a high frequency vibration in response to the current. The interaction between high frequency vibrations with the fluid expanding or inflating the expandable member 30 may create acoustic pressure waves 66 that travel radially outward from the wave generator 40, through the fluid, through the expandable member 30, and to a target location at a vessel wall of a subject. Although the acoustic pressure waves 66 are depicted as being initiated from only two sides of the wave generator 40, the acoustic pressure waves 66 may be initiated at one or more sides of the wave generator 40 and/or circumferentially about the wave generator 40. As discussed, the shape of the lobes 32 with the apex or peak 34 and the valleys 35 and/or the alignment of the wave generator 40 with the apex or peak 34 may direct the acoustic pressure waves 66 through the expandable member 30 at or around the apex or peak 34 of the lobe 32 and amplify forces of the generated acoustic pressure waves 66 to more efficiently modify calcium or plaque build-up at or proximate the target location than when the waves from the wave generators 40 propagate radially outward from wave generators freely (e.g., when one or more wave generators are positioned within an elongate cylindrical balloon).

The current or other power provided to the wave generator 40 may determine an excitation frequency of the piezoelectric transducer 46 and thus, a frequency of the acoustic pressure waves 66. In some examples, the current or other power provided to the wave generator 40 may be applied or provided at a frequency configured to produce waves 66 at a frequency and amplitude sufficient to break-up calcium or plaque at a target site and/or cause a drug coated on an exterior surface of the expandable member 30 to separate from the expandable member 30 and transfer to the target location or vessel wall of the subject. Example suitable frequencies range from 1 kilohertz (kHz) to 100 kHz, from 25 kHz to 100 kHz, and/or may be in one or more other suitable frequency ranges. In one example, the acoustic pressure waves sufficient to break-up calcium or plaque at a target site and/or cause a drug coated on an exterior surface of the expandable member 30 to separate from the expandable member 30 may have a frequency in a range of 23 kHz to 25 kHz, but other suitable frequencies are contemplated.

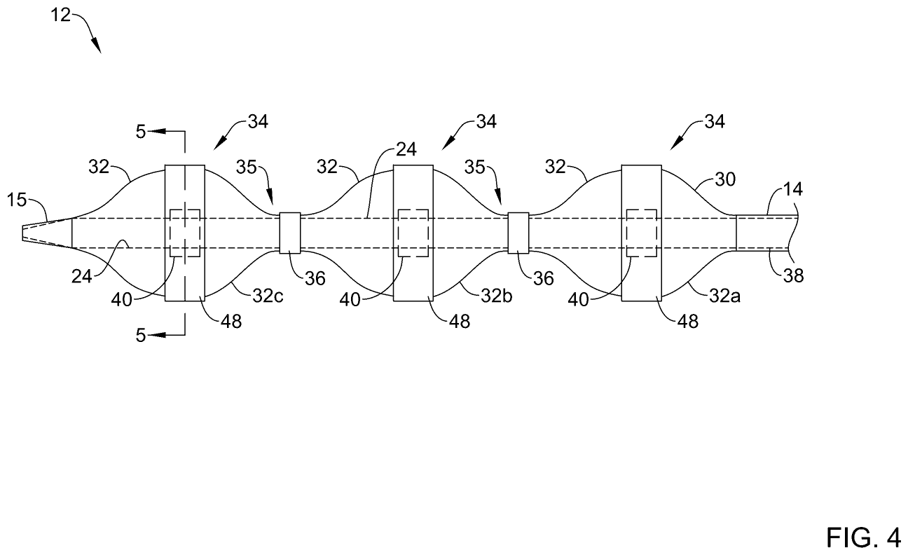

FIG. 4 depicts a side view of an illustrative configuration of the medical device 12. As depicted in FIG. 4, the medical device 12 may include an expandable member 30 with three lobes 32 (e.g., the first lobe 32a, the second lobe 32b, and the third lobe 32c) and a first annular member 36 extending circumferentially around the expandable member 30 between the first lobe 32a and the second lobe 32b and a second annular member 36 extending circumferentially around the expandable member 30 between the second lobe 32b and the third lobe 32c.

One or more of the lobes 32 may include a coating 48 (e.g., a drug coating and/or other suitable coating) applied to the outer surface of the expandable member 30. In some examples, drug coated expandable members may be exposed to frictional forces that occur when navigating the human vasculature within guide catheters. This friction can cause a significant drug loss to the systemic circulation before reaching the target lesion in the coronary artery. Excipients may be used to minimize drug losses but as complete drug transfer to the vessel must be achieved in 30-60 seconds, these excipients can only create a weak bond between drug and expandable member that is easily breakable during delivery and/or upon expandable member expansion. Due to these weak bonds, drug losses remain significant. By using the high frequency acoustic waves through the expandable member as a drug-debonding mechanism, other excipients capable of providing a stronger bond can be used to avoid drug losses and still be able to unbind and transfer drug to the vessel wall in 30-60 seconds. As such, utilizing the acoustic wave as a drug-debonding mechanism may allow for an enhanced drug transfer to a vessel wall that minimizes drug losses and allows for a drug dosage without compensating overages.

The coating 48 may be applied to an entirety of the outer surface of the expandable member 30 or to one or more target locations along one of or each of the lobes 32. In one example and as depicted in FIG. 4, the coating 48 may be applied to an outer surface of the expandable member 30 at or proximate the apex or peak 34 of each of the lobes 32. In some examples, the coating 48 may be longitudinally aligned with the apex or peak 34 of the lobe 32 and/or the wave generator 40 associated with the lobe 32.

When the coating 48 is applied to the lobe 32 at or proximate the apex or peak 34, the coating 48 may be positioned at a location along the expandable member 30 configured to contact a calcified lesions or stenosis in a vessel of the subject, such that that coating 48 may be directly applied to the calcified lesion or stenosis while mitigating an amount of coating that needs to be used. In contrast, when a cylindrical balloon is utilized in an IVL treatment, an entirety or a large portion of the cylindrical portion of the balloon is coated with a coating despite potentially only a portion of the cylindrical portion of the balloon contacting the calcified lesion or stenosis, which may lead to waste or inadvertently applying a drug inconsistently to the vasculature of the subject.

The coating 48 may be any suitable type of coating. For example, an illustrative coating 48 may be an antiproliferative drug or active ingredient, paclitaxel, an excipient i.e., an acetyltributylcitrate (ATBC), a citrate ester, acrylic adhesives, biodegradable polymers, polyactic acid materials (PLA), shellac, sorbitol, urea, iopromide and/or other suitable coatings. In one example, one or more coatings 48 on the expandable member 30 may include an antiproliferative drug or active ingredient and an excipient that may provide a stronger bond between an outer surface of the expandable member 30 and the antiproliferative drug or active ingredient than a bond between the outer surface of the expandable member 30 and the antiproliferative drug or active ingredient created by ATBC (e.g., a light binder) configured to allow the drug or active ingredient to separate from the expandable member 30 independent of a release mechanism. In some examples, the antiproliferative drug or active ingredient may be paclitaxel and the strong-bond excipient may be a PLA material or biodegradable polymer, but other suitable antiproliferative drugs and/or active ingredients are contemplated.

The coating 48 may be applied to the outer surface of the expandable member 30 in any suitable manner. Example suitable manners for applying the coating 48 to an expandable member 30 (e.g., a balloon), example materials for the antiproliferative drug or active ingredient of the coating 48, and example materials for the expandable member 30 configured to receive the coating 48 are disclosed in, but are not limited to, U.S. Pat. No. 9,192,697 B2 filed on Feb. 9, 2011, and titled BALLOON CATHETER FOR TREATING STENOSIS OF BODY PASSAGES AND FOR PREVENTING THREATENING RESTENOSIS, which is hereby incorporated by reference in its entirety for any and all purposes.

Using a strong-bond excipient to couple the antiproliferative drug or active ingredient to the expandable member 30 may facilitate securing the antiproliferative drug or active ingredient to the outer surface of the expandable member 30 to prevent or mitigate the antiproliferative drug or active ingredient from inadvertently separating from the expandable member 30 as the medical device 12 is delivered to a target location within a vessel of the subject. Mitigating unintended separation of the antiproliferative drug or active ingredient and the expandable member may reduce an amount of antiproliferative drug or active ingredient used during a procedure because less of the drug or active ingredient is lost due to waste.

When a strong-bond excipient is utilized to couple the expandable member 30 and the antiproliferative drug or active ingredient, the acoustic pressure waves 66 may be utilized to release the antiproliferative drug or active ingredient from the expandable member 30, as discussed. In some examples, the acoustic pressure waves 66 may be configured to be of such a frequency and/or amplitude at a location of the coating 48 on the expandable member 30 that in response to the acoustic pressure waves 66 contacting the coating 48 the bond of the strong-bond excipient is severed or broken, the antiproliferative drug or active ingredient separates from the expandable member 30 and is released at the target location(s) on the vessel wall of the subject. In one example, acoustic pressure waves 66 configured to treat calcified lesions may be sufficient to sever or break the bond of the strong-bond excipient.

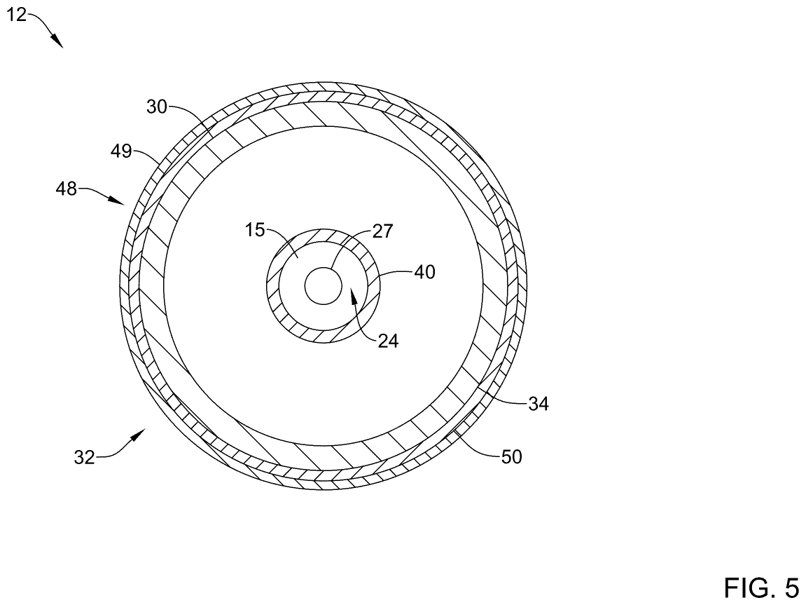

FIG. 5 depicts a schematic cross-section view of the illustrative configuration of the medical device 12 depicted in FIG. 4, taken along line 5-5 extending through the apex or peak 34 of the lobe 32. As depicted in FIG. 5, the coating 48 may include an antiproliferative drug or active ingredient 49 and an excipient 50 coupling the antiproliferative drug or active ingredient 49 with an outer surface of the expandable member 30. In some examples, the excipient 50 may be a strong-bond excipient including but not limited to a PLA material or a biodegradable polymer.

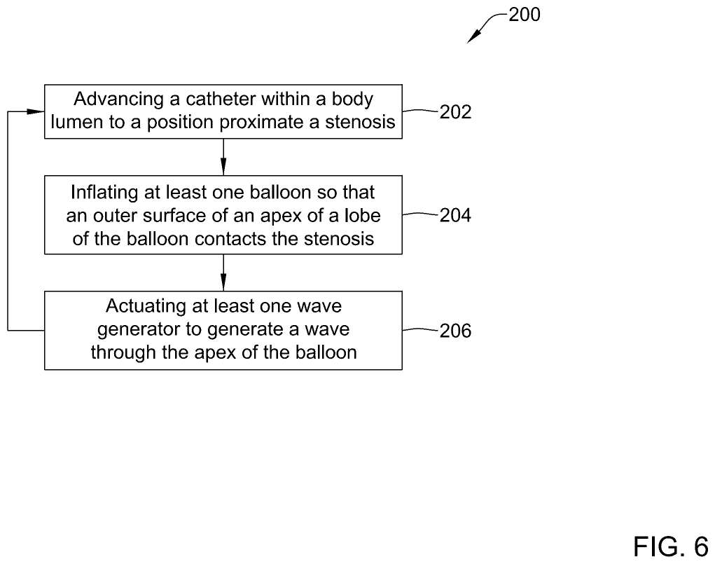

FIG. 6 depicts a schematic diagram of an illustrative method 200 of treating a calcified lesion or stenosis in a body lumen of a subject. The method 200 of treating the calcified lesion or stenosis may utilize a medical device (e.g., a catheter) that includes, an elongate shaft, at least one wave generator, and at least one expandable member (e.g., a balloon) extending around the at least one wave generator as illustrated in FIGS. 1-5. In some examples, the expandable member may have a plurality of lobes that may or may not have an antiproliferative drug or active ingredient coated thereon.

The method 200 may include inserting a medical device (e.g., a catheter) into a body lumen (e.g., a blood vessel) of a subject (e.g., a patient) and advancing 202 the medical device to a position at or proximate a target location within the body lumen. In some examples, the target location may be a calcified lesion or stenosis within the body lumen.

When the medical device has been advanced to the position at or proximate the target location within the body lumen, the one or more expandable members may be expanded or inflated 204. In some examples, the expandable member may be expanded by inserting a fluid through a fluid port and fluid lumen to the interior of the expandable member. The expandable member may be expanded in one or more other suitable manners. When the expandable member includes one or more lobes having an apex or peak and a valley, the expandable member may be expanded such that an outer surface of the apex or peak of at least one of the one of the one or more lobes contacts the target location.

Once the apex or peak of the at least one of the one or more lobes is in contact with the target location, the one or more wave generators may be actuated 206 to generate at least one wave (e.g., acoustic pressure waves) in the fluid expanding or inflating the expandable member and through the fluid and the apex or peak in contact with the target location. The waves that are generated may be amplified by the shape of lobe and propagate through the fluid and the apex or peak of the lobe and into the target location to break up the calcified lesion or stenosis. When the lobe includes a coating, the waves propagating through the expandable member may cause a material bonding an antiproliferative drug or active ingredient to the expandable member to release the antiproliferative drug or active ingredient and transfer the antiproliferative drug or active ingredient to the target location for treating the calcified lesion or stenosis at the target location at the same or similar time as the calcified lesion or stenosis is being debulked.

If there are multiple target locations within the body lumen(s) of the subject, the method 200 may be repeated to treat the multiple target locations sequentially. For example, after treating a first target location, the expandable member may be deflated, the medical device may be advanced 202 longitudinally and/or rotated to a second target location, the expandable member may be inflated 204 at or proximate the second target location, and one or more wave generators may be actuated 206 to generate acoustic pressure waves and apply acoustic pressures waves to the second target location. The first target location and the second target location may be part of the same calcified lesion or stenosis and/or different calcified lesions or stenoses. When the expandable member includes a plurality of lobes, a first lobe of the plurality of lobes may be used to treat a calcified lesion or stenosis at the first target location, a second lobe of the plurality of lobes may be used to treat a calcified lesion or stenosis at the second target location, and so on. In some examples, the lobes may be selectively expanded or inflated relative to one another and/or the wave generators may be selectively actuated relative to one another, but other suitable configurations are contemplated.

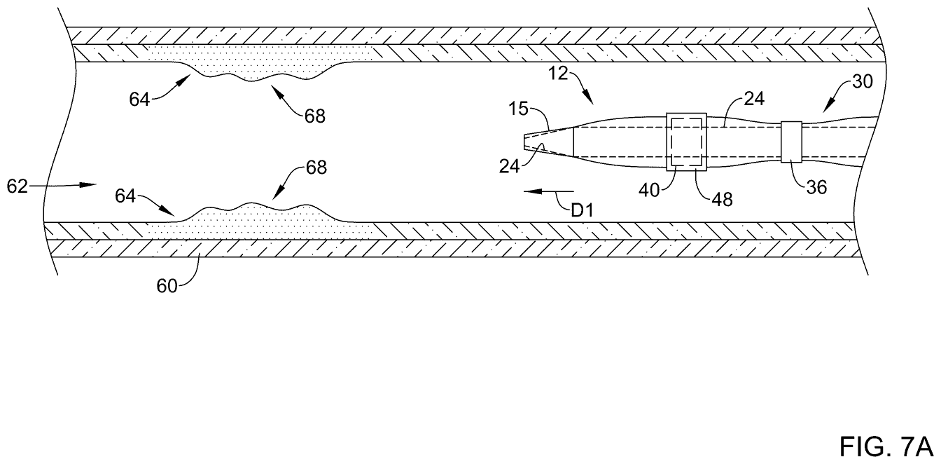

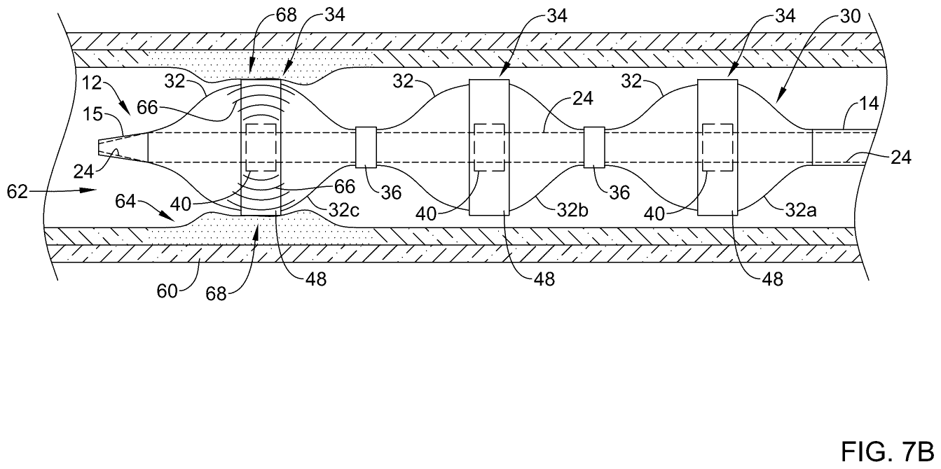

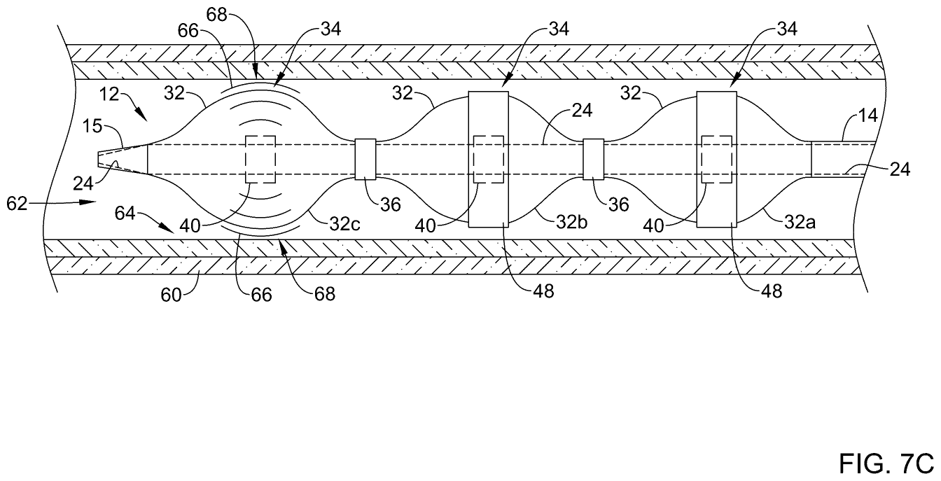

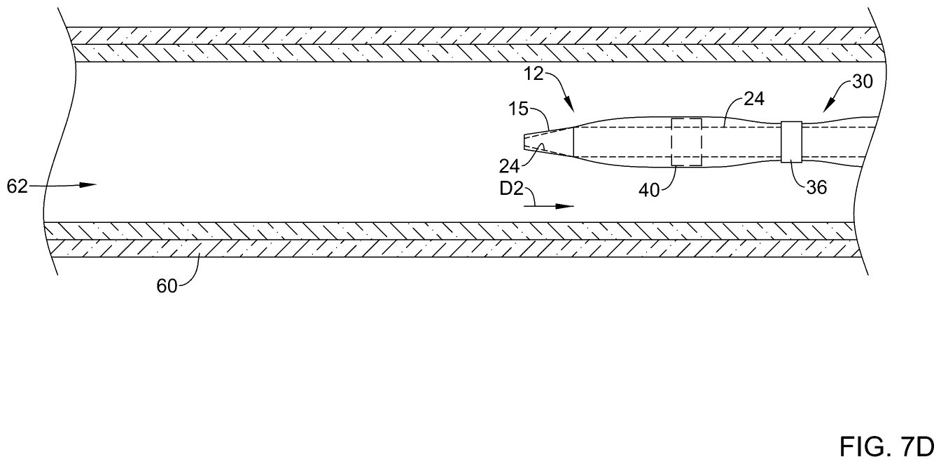

FIGS. 7A-7D depict schematic diagrams of an illustrative method of treating a calcified lesion or stenosis 64 in a blood vessel 60 defining a vessel lumen 62 (e.g., a body lumen). As depicted in FIGS. 7A-7D, the blood vessel 60 and the calcified lesion or stenosis 64 is schematically depicted in cross-section and the medical device 12 is schematically depicted from a side view.

FIG. 7A depicts a schematic view of the medical device 12 being advanced in a first direction D1 (e.g., a distal direction) toward the calcified lesion or stenosis 64. For example, the medical device 12 may be advanced in the first direction D1 until the wave generator 40 associated with one of the lobes 32 is aligned with the target location 68 at the calcified lesion or stenosis 64. Radiographic or magnetic resonance imaging techniques may be used to visualize the location of the medical device 12 in relation to the target location of the calcified lesion or stenosis 64. The medical device 12 may be assembled from and/or may have radiopaque materials attached thereto to aid in visualization.

In at least some configurations, portions or all of the system and/or components thereof may also be doped with, made of, or otherwise include a radiopaque material. Radiopaque materials are understood to be materials capable of producing a relatively bright image on a radiographic (i.e., fluoroscopy) screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some configurations, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

FIG. 7B depicts a schematic view of the medical device 12 with the wave generator 40 associated with the third lobe 32c and the peak or apex 34 of the third lobe 32c aligned with the target location 68, the expandable member 30 expanded or inflated with fluid, and the wave generator 40 associated with the third lobe 32c initiating acoustic pressure waves 66 in the fluid in the expandable member 30. In some examples, acoustic pressure waves 66 may disengage the coating 48 (e.g., an antiproliferative drug or active ingredient) from the third lobe 32c and transfer the coating 48 the to the target location 68 to treat the calcified lesion or stenosis 64. Although FIG. 7B depicts all of the lobes 32 of the expandable member 30 expanded or inflated, it is contemplated that the lobes 32 may be individually expanded or inflated. Although FIG. 7B depicts the acoustic pressure waves 66 being initiated only from the wave generator 40 associated with the third lobe 32c, it is contemplated that all of the wave generators 40 or two or more of the wave generators 40 may be actuated simultaneously or individually to initiate acoustic pressure waves 66 from those wave generators 40.

FIG. 7C depicts a schematic view of the third lobe 32c at the target location 68 after the acoustic pressure waves 66 have been applied to the target location for a period of time. The period of time may be preset or determined during the procedure based on the effectiveness of the treatment and/or one or more parameters. Further, the coating 48 has been released from the third lobe 32c and applied to the blood vessel 60 in response to the acoustic pressure waves 66 disengaging the coating 48 from the third lobe 32c. In some examples and as depicted in FIG. 7C, the coating 48 may remain on the lobes 32 (e.g., the first lobe 32a and the second lobe 32b) having wave generators 40 that were not actuated.

FIG. 7D depicts a schematic view of the medical device 12 being advanced in a second direction D2 (e.g., a proximal direction) after treating the calcified lesion or stenosis at the target location 68. In some examples, the medical device 12 may be advanced in the second direction D2 to remove the medical device from the subject and/or to longitudinally adjust the medical device to a location at which the wave generator 40 associated with one of the lobes 32 (e.g., the first lobe 32a or the second lobe 32b) is aligned with a second target location 68. Although the medical device 12 is being longitudinally advanced in the second direction D2, the medical device 12 may be longitudinally advanced in the first direction D1 to a further target location 68.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

The materials that can be used for the various components of the medical device 12, and the various elements thereof disclosed herein may include those commonly associated with medical devices. For simplicity purposes, the following discussion refers to the assembly. However, this is not intended to limit the devices and methods described herein, as the discussion may be applied to other elements, members, components, or devices disclosed herein, such as, but not limited to, the elongate shaft, the annular member, the hub, the distal tip, the ports, the lumens, and/or elements or components thereof.

In some embodiments, the device and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

What is claimed is:

1. A medical device, the medical device comprising:
an elongate shaft;
at least one wave generator; and
at least one expandable member extending around the at least one wave generator,
wherein the at least one expandable member has a plurality of lobes including a first lobe and a second lobe longitudinally spaced from the first lobe.

2. The medical device of claim 1, wherein the at least one expandable member has a sinusoidal shape when inflated.

3. The medical device of claim 1, wherein a wave generator of the at least one wave generator is longitudinally aligned with an apex of one of the first lobe and the second lobe.

4. The medical device of claim 1, wherein a first wave generator of the at least one wave generator is longitudinally aligned with a first apex of the first lobe and a second wave generator of the at least one wave generator is longitudinally aligned with a second apex of the second lobe.

5. The medical device of claim 1, further comprising:
a ring extending circumferentially around the at least one expandable member between the first lobe and the second lobe, the ring having an outer diameter that is less than an outer diameter of the first lobe, an outer diameter of the second lobe, or the outer diameter of the first lobe and the outer diameter of the second lobe.

6. The medical device of claim 1, further comprising:
a first ring extending circumferentially around the at least one expandable member;
a second ring extending circumferentially around the at least one expandable member;
a third lobe of the plurality of lobes, the third lobe being longitudinally spaced in a distal direction from the first lobe and the second lobe, and
wherein the first ring extends circumferentially around the at least one expandable member between the first lobe and the second lobe, and
wherein the second ring extends circumferentially around the at least one expandable member between the second lobe and the third lobe.

7. The medical device of claim 1, wherein the first lobe is in fluid communication with the second lobe.

8. The medical device of claim 1, further comprising:
a fluid lumen extending along the elongate shaft, the fluid lumen being in fluid communication with the expandable member.

9. The medical device of claim 8, wherein the fluid lumen is in fluid communication with each of the plurality of lobes.

10. The medical device of claim 1, wherein a wave generator of the at least one wave generator comprises a piezoelectric transducer.

11. The medical device of claim 1, further comprising:
an antiproliferative drug coupled with an exterior surface of one or more of the plurality of lobes.

12. The medical device of claim 11, wherein the antiproliferative drug is coupled with the exterior surface of the at least one expandable member using a biodegradable polymer.

13. A system comprising:
a catheter comprising:
an elongate shaft;
at least one wave generator; and
at least one expandable member extending around the at least one wave generator, and
wherein the at least one expandable member comprises a sinusoidal shape when inflated; and
a power generator configured to actuate the at least one wave generator to initiate a wave within the at least one expandable member.

14. The system of claim 13, wherein the power generator is configured to apply an alternating current to the at least one wave generator.

15. The system of claim 13, wherein the at least one expandable member is coated with an antiproliferative drug bound to an outer surface of the at least one expandable member with an excipient.

16. The system of claim 15, wherein the power generator is configured to apply an alternating current to the at least one wave generator at a frequency that causes the wave generated by the at least one wave generator to detach the antiproliferative drug from the outer surface of the at least one expandable member.

17. The system of claim 13, wherein the catheter comprises a fluid lumen configured to provide fluid to the at least one expandable member.

18. A method for treating a stenosis in a body lumen, the method comprising:
advancing a catheter within the body lumen to a position proximate to the stenosis, the catheter comprising an elongate shaft, at least one wave generator, and at least one expandable member extending around the at least one wave generator and having a plurality of lobes;
inflating the at least one expandable member with a fluid so that an outer surface of an apex of at least one of the plurality of lobes contacts the stenosis; and
actuating the at least one wave generator to generate at least one wave through the fluid and the apex of the at least one of the plurality of lobes.

19. The method of claim 18, wherein the at least one expandable member is coated with an antiproliferative drug, and wherein the at least one wave generated by the at least one wave generator is configured to detach the antiproliferative drug from the at least one expandable member.

20. The method of claim 18, further comprising:
longitudinally adjusting the catheter to treat different portions of the stenosis sequentially.
